(19) United States
(12) Patent Application Publication
(10) Pub. No.: US 2007/0021680 A1
(43) Pub. Date: Jan. 25, 2007

(54) Methods to reduce power to measure pressure

(75) Inventor: Perry A. Mills, Arden Hills, MN (US)

Correspondence Address: FOGG AND ASSOCIATES, LLC, P.O. BOX 581339, MINNEAPOLIS, MN, 55458-1339, US

(73) Assignee: Transoma Medical, Inc., St. Paul, MN (US)

(21) Appl. No.: 11/187,640

(22) Filed: Jul. 22, 2005

(51) Int. Cl. A61B 5/04; A61B 5/117; A61B 5/103; A61B 5/02; A61M 31/00

(52) U.S. Cl. 600/522; 600/483; 604/67; 600/486; 600/510; 600/513; 600/521; 600/587; 600/591

(57) ABSTRACT

Methods and systems for reducing power in pressure monitoring devices are provided. The method includes monitoring a physiological function, detecting a need for an adjustment in therapy, and qualifying the need for an adjustment in therapy. Qualifying the need for an adjustment in therapy includes transmitting a signal requesting a pressure measurement based on the detected need for an adjustment in therapy, applying power to a pressure sensor and measuring pressure. The method further includes adjusting therapy when the measured pressure confirms the detected need for an adjustment in therapy.

### TECHNICAL FIELD

The present invention relates to the reduction of power in monitoring physiological pressures, including blood pressure, intracranial pressure, intrapleural pressure, uterine pressure, and pressure within the gastrointestinal system and in particular blood pressure monitoring and blood pressure measurement combined with therapy.

### BACKGROUND

The reduction of power required for the measurement of physiological pressures is desirable for stand alone pressure monitoring devices as well as devices combining pressure monitoring with therapy. Power limitations are particularly severe for multifunction devices designed for chronic implant where very little power can be allocated to the pressure measurement function. An example of such a device would be a Left Atrial (LA) or Left Ventricular (LV) pressure monitor used to assess hemodynamic status of a heart failure patient in order to guide electrophysiological, pharmacological, or other therapy. This therapy may be delivered by an implantable device such as a pacemaker, defibrillator, or drug infusion pump, which could also house and power the pressure measurement capability. Here, the desire for a small device (which requires a small battery) and the multifunction nature of the device leave only a small power budget for the pressure measurement function.

In particular, the reduction in power requirements of sensors and circuitry used to measure blood pressure is needed. This need arises from applications such as monitoring of blood pressure in order to regulate cardiac therapy such as pacing or defibrillation. A defibrillator, for example, may have a battery current drain under 10 μA allowing a battery life in excess of 5 years. This 10 μA current drain is expended primarily in monitoring and processing ECG to assess whether the heart is fibrillating. It would be desirable to add the capability of monitoring blood pressure to include heart hemodynamics in the assessment of fibrillation. To add this capability while retaining a battery life close to 5 years would require new methods to reduce the power requirements needed for pressure monitoring.

This application describes systems and methods to conserve power required for pressure measurement under these and other circumstances.

For the reasons stated above, and for other reasons stated below which will become apparent to those skilled in the art upon reading and understanding the present specification, there is a need in the art for reducing power required for pressure measurements.

### SUMMARY

The above-mentioned problems as well as other problems are addressed by embodiments of the present invention and will be understood by reading and studying the following description.

In one embodiment, a method of reducing power in a pressure monitoring device is provided. The method includes monitoring an electrocardiogram (ECG) waveform, detecting a fiduciary point in the ECG waveform, and transmitting a signal requesting a pressure measurement, after a predefined delay. The predefined delay is determined based on the desired pressure measurement and the detected fiduciary point. The method further includes applying power to a pressure sensor and applying an output voltage of the pressure sensor to a sampling circuit. The output voltage represents measured pressure.

In one embodiment, a method of reducing power in a therapy device is provided. The method includes monitoring a physiological function, detecting a need for an adjustment in therapy and qualifying the need for an adjustment in therapy. Qualifying the need for an adjustment in therapy includes transmitting a signal requesting a pressure measurement based on the detected need for an adjustment in therapy, applying power to a pressure sensor and measuring pressure. The method further includes adjusting therapy when the measured pressure confirms the detected need for an adjustment in therapy.

In one embodiment, an implantable device is provided. The device includes a monitoring device configured to measure pressure for the detection of one or more heart related disorders and includes a pressure sensor and a sampling capacitor coupled to an output of the pressure sensor and adapted to charge to a voltage output that represents the mean pressure. The pressure sensor is strobed at a rate to create a low-pass filter effect rate mean pressure.

In one embodiment, a pressure monitoring device is provided. The device includes a means for monitoring an electrocardiogram (ECG) waveform, a means for detecting a fiduciary point in the ECG waveform, a means for transmitting a signal requesting a pressure measurement, after a predefined delay, a means for applying power to a pressure sensor and a means for applying an output voltage of the pressure sensor to a sampling circuit. The predefined delay is determined based on the desired pressure measurement and the detected fiduciary point. The output voltage represents measured pressure.

### BRIEF DESCRIPTION OF THE DRAWINGS

The present invention can be more easily understood and further advantages and uses thereof more readily apparent, when considered in view of the description of the preferred embodiments and the following figures in which:

FIG. 1 is a flow chart of one embodiment of a method of reducing power for pressure measurement according to the teachings of the present invention.

FIG. 2 is a flow chart of another embodiment of a method of reducing power for pressure measurement according to the teachings of the present invention.

FIG. 3 is a block diagram of one embodiment of a pressure measurement system according to the teachings of the present invention.

FIG. 4 is a block diagram of another embodiment of a pressure measurement system according to the teachings of the present invention.

FIG. 5 is one embodiment of an ECG waveform in relation to a corresponding pressure waveform.

FIG. 6 includes an additional graph showing a pressure waveform and an ECG waveform plotted along a common time axis.

In accordance with common practice, the various described features are not drawn to scale but are drawn to emphasize specific features relevant to the present invention.

### DETAILED DESCRIPTION

In the following detailed description, reference is made to the accompanying drawings that form a part hereof, and in which is shown by way of illustration specific illustrative embodiments in which the invention may be practiced. These embodiments are described in sufficient detail to enable those skilled in the art to practice the invention, and it is to be understood that other embodiments may be utilized and that logical, mechanical and electrical changes may be made without departing from the spirit and scope of the present invention. The following detailed description is, therefore, not to be taken in a limiting sense.

Embodiments of the present invention provide systems and methods to reduce the power requirements of sensors and circuitry used to measure physiological pressure. Much of this power is consumed by the pressure sensor itself or the circuitry needed to amplify and digitize the signal from the sensor. In one embodiment, both the sensor and the circuitry are typically strobed (powered intermittently) each time pressure is sampled to consume power only when necessary. The average power required is then proportional to the rate that samples are acquired and the duration that power is applied to the sensor or measurement circuits for each sample.

One method of strobed blood pressure sample measurement consists of applying power to a pressure sensor, applying the sensor output voltage to a sample and hold (S&H) capacitor, and disconnecting the S&H capacitor from the sensor and removing power to the sensor once the voltage on the S&H capacitor is stable. Power efficiency of this strobing technique is improved by reducing the pulse width of the strobe. In one embodiment, this pulse width is reduced by placing an active voltage buffer after the pressure sensor output to reduce the impedance driving the S&H capacitor. This reduced impedance charges the S&H capacitor to a stable voltage more quickly and allows a shorter strobe pulse.

In another embodiment, pulse width is reduced by decreasing the capacitance of the S&H capacitor. The capacitance value is often chosen to be high enough to control measurement error due to charge leakage from the capacitor or due to changes in parasitic capacitance. Both the charge leakage and parasitic capacitance affects are reduced by including the capacitor and associated circuitry and switches on an Integrated Circuit (IC) rather than by using a discrete component approach. The charge leakage can be reduced further by migrating to a less leaky IC process, adding components that provide an equal but opposite compensating leakage, or reducing the period of time the sample is held on the S&H capacitor (hold time). Hold time can be minimized by using a fast Analog-to-Digital conversion instead of a slower method such as converting voltage to time with a voltage ramp and a voltage comparator.

In one embodiment, the pressure sensor is a resistive bridge type such as a silicon piezoresistive sensor although these concepts may be applied to a variety of other types of pressure sensors. The bridge output resistance of pressure sensors is often in the 300 to 10000 ohms range as constrained by sensor size, sensitivity, and stability. Given this range of resistance it is important to minimize the duration that the pressure sensor is powered to be as short as possible.

In one embodiment, pressure measurement power is reduced by acquiring pressure samples of the pressure waveform only during specific points or segments of the cardiac pressure waveform.

Embodiments of the present invention reduce the power required to monitor blood pressure by one or both of sampling only selected points in the cardiac cycle as identified by another parameter such as ECG and monitoring only when another parameter such as ECG has identified a potential problem such as fibrillation or tachycardia. In one embodiment, blood pressure is monitored briefly to confirm the diagnosis in order to avoid inappropriate therapy such as delivery of defibrillation shocks.

In addition, embodiments of the present invention reduce the power required to monitor blood pressure by reducing the duration of each strobe of the pressure sensor to obtain mean pressure. In one embodiment, a shorter strobe duration is used to produce a low-pass filter effect to generate mean pressure. In an alternate embodiment, sampling is performed in two stages with a sampling capacitor and a low value transfer capacitor.

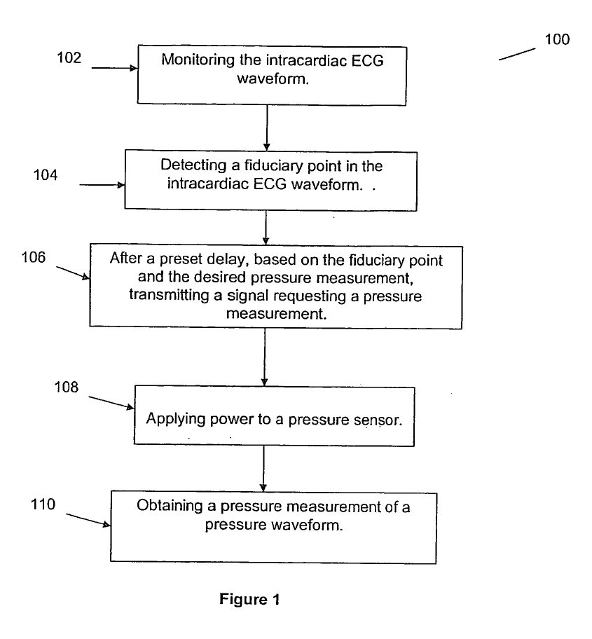

FIG. 1 is a flow chart for one embodiment of a method for reducing power for pressure measurements, shown generally at 100 according to the present invention. It is understood that for illustration purposes this method is discussed with respect to blood pressure measurements but any physiological pressure may be employed. Block 102 of method 100 monitors the intracardiac ECG waveform. The method proceeds to block 104 and detects a fiduciary point in the intracardiac waveform. In one embodiment, instead of monitoring blood pressure and reproducing the waveform by taking multiple measurements the power required to accomplish this is reduced by taking defined measurements based from a fiduciary point. For example, embodiments involving pressure measurement devices such as a blood pressure monitor may request a pressure measurement based on a fiduciary point of an intracardiac ECG waveform sensed by the device.

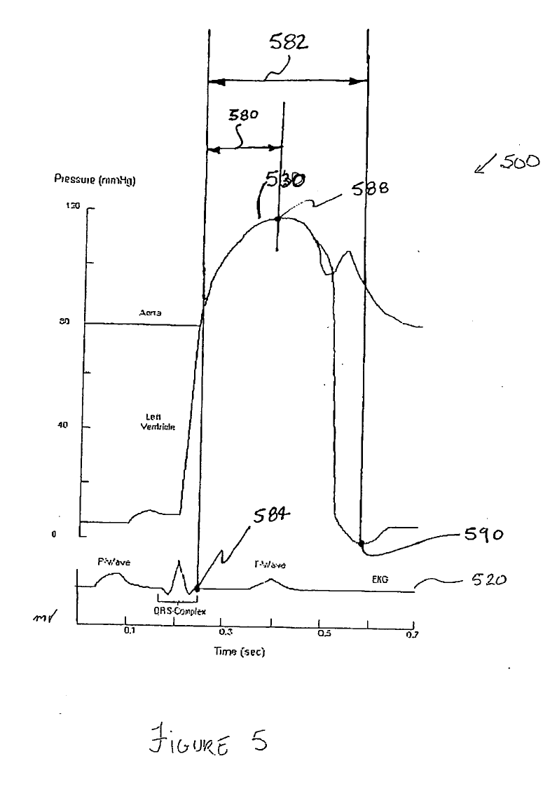

In one embodiment, the fiduciary point is the QRS complex of the ECG waveform. In another embodiment, the fiduciary point is the P, Q, R, S, or T wave of the ECG waveform or another point. See the graphs of FIG. 5. FIG. 5 provides one embodiment of a graph having an intracardiac ECG waveform 520 plotted along a time axis with a corresponding pressure waveform 530 plotted along the same time axis and a pressure axis. The amplitude of ECG waveform 520 is along a voltage axis in millivolts. In one embodiment, the fiduciary point is an end point of one of the P, Q, R, S, and T waveforms.

The method proceeds to block 106 and after a preset delay transmits a signal requesting a pressure measurement. The preset delay is based from the fiduciary point and determined based on the type of measurement needed for a specific therapy. In one embodiment, the delay is chosen to approximate the point in time that maximum pressure occurs. In another embodiment, a predetermined delay is chosen to acquire minimum pressure. In some embodiments, these maximum and minimum pressures correspond to systolic pressure and diastolic pressure based on the definitions applied. In one embodiment, for both maximum and minimum pressure, the appropriate delay from a fiduciary point such as from the QRS complex are modified based on heart rate as determined from recent intervals of the ECG waveform, for the particular patient.

In one embodiment, the fiduciary point is an endpoint of a QRS complex and the predefined delay comprises between about 0.16 seconds and about 0.56 seconds. In one embodiment, the predefined delay is between about 0.26 seconds and about 0.46 seconds. In one embodiment, the predefined delay comprises about 0.36 seconds.

In one embodiment, the fiduciary point comprises a peak of an R wave and the predefined delay is between about 0.18 seconds and about 0.58 seconds. In one embodiment, the predefined delay is between about 0.28 seconds and about 0.48 seconds. In one embodiment, the predefined delay is about 0.38 seconds.

In one embodiment, the appropriate delays are modified based on occasional monitoring of the pressure waveform during one or more preceding cardiac cycles to locate the position of the actual minimum, maximum, or other pressure points relative to ECG waveform fiduciary points. In one embodiment, for end-diastolic pressure a longer delay is used from the previous QRS complex or a very short delay is used from any of the immediate P, Q, R, S waves.

In one embodiment, blood pressure monitoring is used to detect and/or monitor hypertension, syncope, congestive heart failure and the like.

In one embodiment, instead of monitoring blood pressure and reproducing the waveform by taking multiple measurements the power required to accomplish this is reduced by taking defined measurements based from a fiduciary point. For example, embodiments involving pressure measurement devices such as a blood pressure monitor may request a pressure measurement based on an intracardiac ECG waveform sensed by the device.

In another embodiment, multiple pressure measurements are obtained that provide full disclosure of the pressure waveform versus one aspect of the pressure waveform. The number and type of measurements are adaptable to the type of monitoring.

Method 100 proceeds to block 108 and power is applied to a pressure sensor based on the request. The method then proceeds to block 110 and obtains one or more pressure measurements based on the specific application.

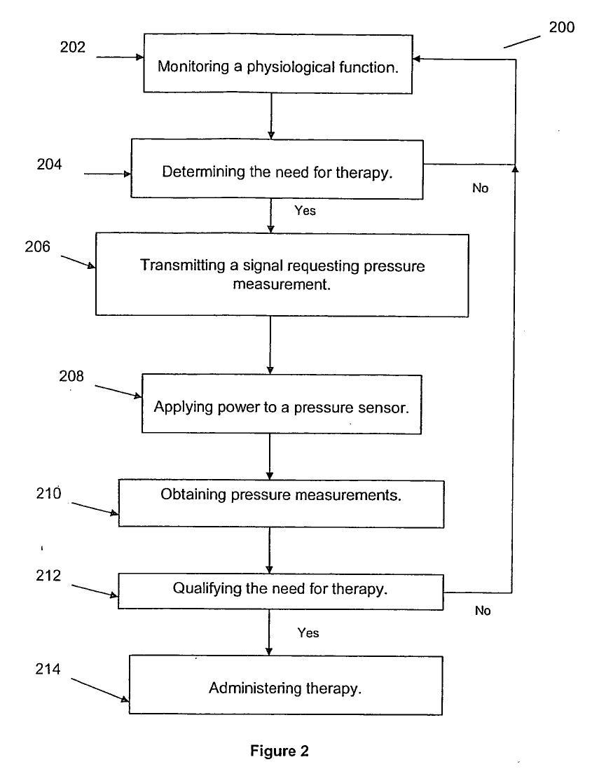

FIG. 2 is a flow chart for one embodiment of a method for reducing power for pressure measurements, shown generally at 200 according to the present invention. In one embodiment, pressure measurement power is reduced for therapy devices combined with pressure monitoring by acquiring pressure samples of the pressure waveform only after monitoring of a physiological function such as intracardiac ECG or another parameter which anticipates a therapy adjustment. In one embodiment, the therapy is a defibrillator shock, change in pacing rate or the like.

Block 202 of method 200 monitors a physiological function such as the intracardiac ECG waveform. The method proceeds to block 204 and determines the need for therapy. If therapy is not required, the method returns to block 202 and continues to monitor the physiological function. When the need for therapy is determined, the method proceeds to block 206 and transmits a signal requesting pressure measurement. The method proceeds to block 208 and power is applied to a pressure sensor. The method proceeds to block 210 and obtains one or more pressure measurements. The method proceeds to block 212 and the need for therapy is qualified based on the obtained pressure measurements. If the pressure measurements confirm the need for therapy the method proceeds to block 214 and appropriate therapy is administered. If the pressure measurements refute the need for therapy, the method proceeds to block 202 and continues monitoring a physiological function. In one embodiment, therapy is used to prevent fibrillation and arrhythmia.

In one embodiment, pressure measurement is utilized to confirm the need for therapy by looking at a full wave form. In one embodiment, for example, when the ECG indicates that fibrillation is occurring, pressure is then monitored briefly to confirm the existence of fibrillation to avoid the delivery of inappropriate defibrillation shocks to the patient. The indication that fibrillation is occurring could be false for example, due to noise, improper lead placement or other error. In this embodiment, significant power is conserved by confirming the need for therapy using pressure measurement versus unnecessarily shocking the patient. In this embodiment, it is assumed that the ECG or other parameter can be continuously monitored for less power than blood pressure can be continuously monitored.

In one embodiment, the ECG waveform is used to identify beginning and end points of the cardiac cycle such that mean pressure is calculated over individual cardiac cycles rather than as a continuous running mean over many cardiac cycles. This provides a more immediate result, which is important in certain types of cardiac therapy such as defibrillation. In one embodiment, the mean pressure is estimated using a predetermined weighted average of the systolic and diastolic pressures as located by keying off the ECG waveform. In one example, mean pressure is calculated using Equation 1.
mean pressure=(2*diastolic +1*systolic)/3) EQUATION 1.

In one embodiment, the mean pressure is calculated by charge sharing of appropriately sized capacitors or other methods. When appropriate it may be preferable to use diastolic or systolic pressure directly.

In one embodiment, analysis of the blood pressure waveform is improved by establishing a pressure baseline from previous (normal physiology) data. This pressure baseline may be needed to detect a change in blood pressure as an indication for therapy delivery or adjustment. The baseline is necessary for example, for absolute pressure without ambient correction or when a sensor is drifting enough to require compensation.

A number of methods to establish a pressure baseline may be used. The approach used will depend on whether the pressure sensor is differential or absolute.

In one embodiment, a differential pressure sensor monitors a pressure difference such as the difference in pressure between blood pressure within a point in the circulatory system and a reference pressure such as abdominal cavity, thoracic cavity, or ambient pressure beneath or through the skin. In each case, the intent is to use a reference pressure that is close to the ambient pressure external to the patient. Since the differential pressure already has ambient pressure subtracted, it may be used to directly regulate therapy if an appropriate time-independent pressure threshold can be determined. If a time-relative pressure threshold is preferred, the differential sensor can also be used to establish a baseline and then measure a time-dependent change from that baseline.

In one embodiment, an absolute pressure sensor measures pressure relative to an internal vacuum reference. Absolute pressure can be used to directly monitor time-relative changes in blood pressure or, if ambient pressure is subtracted, can be used to compare to a time-independent threshold. Detection of relative changes in blood pressure would need to reject changes in blood pressure caused by rapid changes in ambient pressure such as with motion in an elevator or changes in pressurization of an aircraft. If the intent is to measure relative changes in blood pressure, then prior history of blood pressure is needed to compare to the pressure measurement triggered by an ECG event. In one embodiment, this prior history is obtained by occasional monitoring of blood pressure cardiac cycles or by selected samples of blood pressure during each cardiac cycle. One way of using an absolute sensor without a prior pressure history or ambient pressure information would be to use relative pressure information within a cardiac cycle such as pulse pressure or max dP/dT to regulate therapy.

It is understood that for illustration purposes this method is discussed with respect to blood pressure measurements but any physiological pressure may be employed.

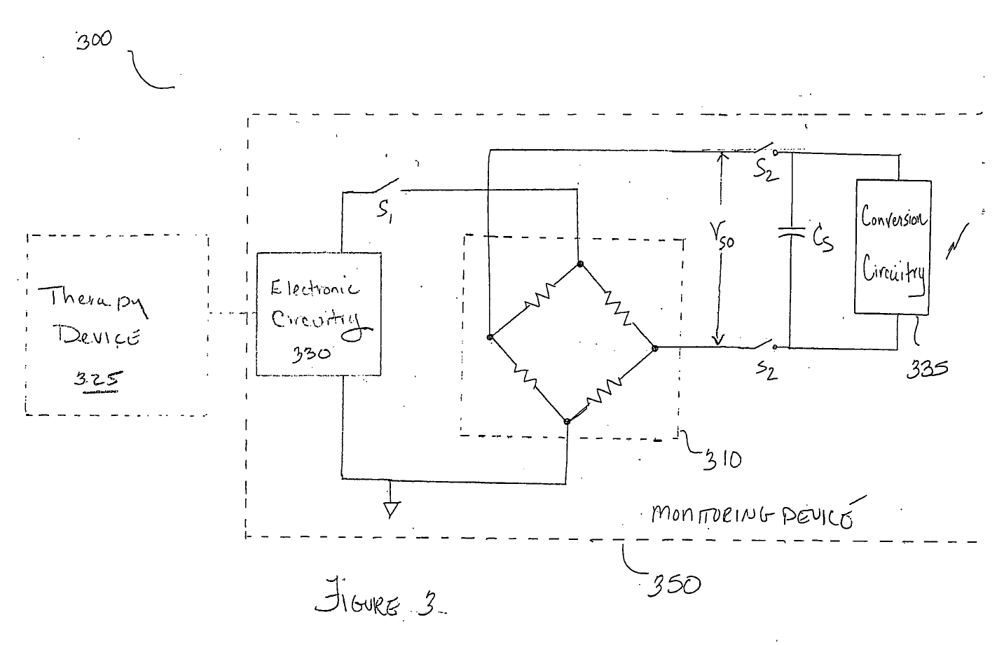

FIG. 3 is a block diagram of one embodiment of an implantable device, shown generally at 300, according to the teachings of the present invention. In one embodiment, device 300 is an implantable monitoring device 350 and includes electronic circuitry 330, a first switch, S₁, coupled between electronic circuitry 330 and pressure sensor 310. Pressure sensor 310 is powered intermittently via electronic circuitry 330. First switch S₁ turns on and off power to pressure sensor 310. Monitoring device 350 further includes sampling capacitor Cs coupled to an output of pressure sensor 310 and Cs charges to pressure sensor 310's output voltage Vso. A second switch S₂ is coupled between capacitor Cs and pressure sensor 310 and aids in preventing charge leakage when the power to pressure sensor 310 is removed. Monitoring device 350 further includes conversion circuitry 335 coupled to the sampling capacitor Cs.

In this embodiment, pressure sensor 310 is illustrated as a piezoresistive bridge type pressure sensor, such as a silicon piezoresistive sensor or the like, although other types of sensors may be employed. In one embodiment, the output resistance of pressure sensor 310 is approximately between 300 to 10000 Ohms and is constrained by the sensor size, sensitivity, stability and the like for a particular application.

In one embodiment, implantable monitoring device 350 is used to monitor intracardiac blood pressure. In another embodiment, implantable monitoring device 350 is used to monitor vascular blood pressure. In one embodiment, monitoring device 350 is used to monitor blood pressure for the detection and/or treatment of hypertension, syncope, congestive heart failure and the like. In an alternate embodiment, implantable device 300 further includes an implantable therapy device 325. In one embodiment, implantable therapy device 325 is a pacemaker, defibrillator, drug infusion pump or the like. In one embodiment, in combination, monitoring device 350 qualifies the need for therapy by measuring a relative parameter of blood pressure. For example, in one embodiment therapy device 325 is a defibrillator and when therapy device 325 determines that the heart needs defibrillating the monitoring device 350 confirms the presence of an aberrant heart rhythm by measuring a relative blood pressure parameter. When both the therapy device 325 and the monitoring device 350 indicate aberrant heart rhythm, therapy is administered.

In one embodiment, to reduce the power required for pressure measurement function, the required pressure information is a reduced bandwidth derivative of an LA or LV waveform such as mean, systolic or diastolic pressure. The average pressure in an arterial system is of interest because it represents the force that is effective throughout the cardiac cycle for driving blood to the tissues. This force is called the mean arterial pressure, and herein referred to as mean pressure.

In some instances the frequency content or bandwidth of the pressure waveform is greater than the bandwidth of the pressure information required to guide therapy. In some embodiments, mean pressure may be all that is required for monitoring pressure for detection and/or therapy. Mean pressure cannot be measured simply by sampling the pressure waveform at a slower rate since the higher frequency content of the pressure waveform may cause error referred to as aliasing. In one embodiment, the duration of each strobe of pressure sensor 310 is reduced to achieve mean pressure in. a way that conserves power. In some systems, the pressure sensor strobe duration is increased to give adequate time for the sensor output resistance to charge sampling capacitor Cs to the full value of the sensor output voltage Vso. This duration increases with both bridge output resistance and the value of the sampling capacitor Cs. In some systems, the sampling capacitor Cs value may be limited from being lower by requirements involving noise, hold time as limited by leakage, or signal amplification. By measuring mean pressure the strobe duration is intentionally shortened.

In one embodiment, when only mean pressure is desired, the strobe duration is intentionally shortened by approximately 70%. In operation sampling capacitor Cs is charged directly by the pressure sensor bridge of pressure sensor 310. The shorter strobe duration does not allow sampling capacitor Cs to charge to the full value of the pressure sensor output voltage Vso. Over multiple samples, this effect creates a desired low-pass filter effect to generate mean pressure. In one embodiment, the strobe duration in 0.1 microsecond, the strobe interval is 50 milliseconds, the pressure sensor output resistance is 10 Kohm, and Cs is 1000 pF. With these parameters the time constant is approximately 5 seconds resulting in a low pass filter 3 dB corner of approximately 0.032 Hz. To achieve a lower frequency filter corner or to allow use of a smaller capacitor, resistance may be added in series with the output of the pressure sensor. When the strobe duration is made short enough to leave only mean pressure without the higher frequency content, then the sampling capacitor charge may be measured or digitized at a relatively low rate by conversion circuitry 335 that also saves power. In operation conversion circuitry 335 amplifies and digitizes output voltage Vso for comparison to a reference voltage. This embodiment prefers that the conversion circuitry 335 will measure but not modify the voltage on capacitor Cs so as to not affect the ongoing generation of the mean pressure indication at Cs. In one embodiment, for monitoring device 350 without the combination of a therapy device the comparison is used to detect any irregularities in the pressure. In another embodiment, for monitoring device 350 in combination with therapy device 325 the comparison is used to qualify the need for therapy.

In one embodiment, conversion circuitry 335 records data collected such as the value of the voltage or charge of sampling capacitor Cs and stores it in memory. In another embodiment, conversion circuitry 335 wirelessly transmits data collected to remote circuitry for analysis andlor recording.

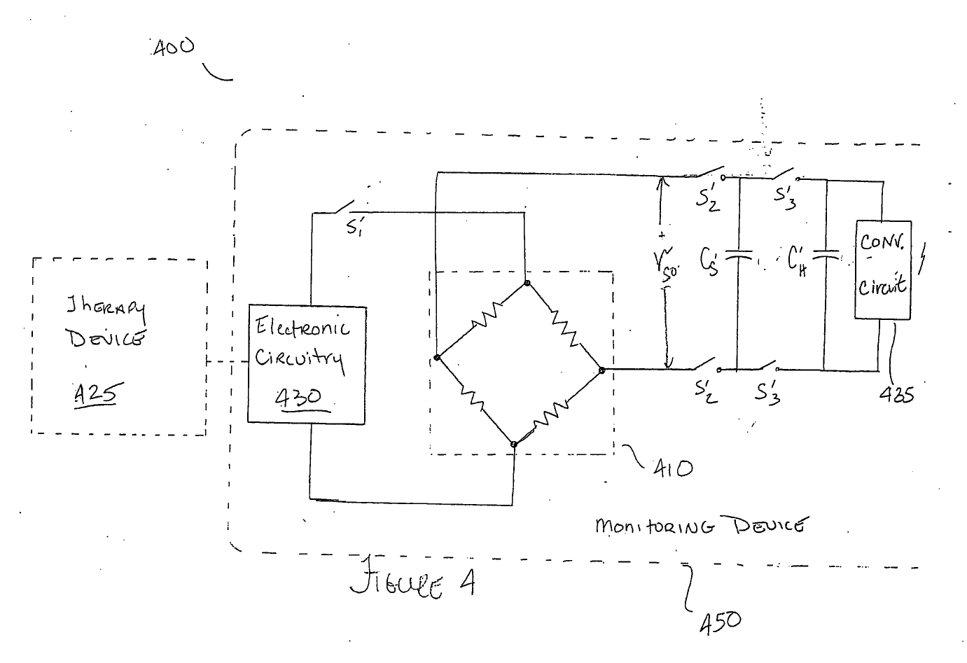

FIG. 4 is a block diagram of another embodiment of an implantable device, shown generally at 400, according to the teachings of the present invention. In one embodiment, device 400 is an implantable monitoring device 450 as described above with respect to FIG. 3. In contrast, implantable monitoring device 450 includes a low value sampling capacitor Cs′ that charges to full value when the output voltage V'so of pressure sensor is applied. In this embodiment, implantable monitoring device 450 includes switch S₁′ that when engaged provides power to pressure sensor 410. In one embodiment, pressure sensor 410 is intermittently powered. Monitoring device 450 further includes a larger value hold capacitor Cₕ′ coupled in parallel to sampling capacitor Cs′ that receives a charge from sampling capacitor Cs′. A second switch S₂′ is coupled between sampling capacitor Cs′ and pressure sensor 410 and aids in preventing charge leakage of Cs′ when power to pressure sensor 410 is removed. A third switch S₃′ is coupled between Cs′ and Cₕ′ and enables the transfer of charge from Cs′ to Cₕ′ and aids in preventing charge leakage of Cₕ′. Monitoring device 450 further includes conversion circuitry 435 coupled to hold capacitor Cₕ′.

In one embodiment, in operation, sampling is accomplished in two stages with a small value sampling capacitor Cs′ and a larger value hold capacitor Cₕ′. In one embodiment, low value sampling capacitor Cs′ is fully charged to the pressure sensor output voltage Vso′ within a much shorter strobe duration and then connected in parallel with transfer capacitor Cₕ′ to again create a low-pass filter effect for the voltage on the sampling capacitor Cs′. As a result, mean pressure is obtained while using less power.

In this embodiment, pressure sensor 410 is illustrated as a piezoresistive bridge type pressure sensor such as a silicon piezoresistive sensor although other types of sensors may be employed. In one embodiment, the conversion circuit 435 samples the voltage on Cₕ′ with a high impedance buffer or amplifier to avoid modifying the charge on Cₕ′ and affecting subsequent measurements of mean pressure. System 400 enables achieving mean pressure in a way that conserves power. In contrast to previous pressure measurement systems, where the pressure sensor strobe duration is increased to give adequate time for the sensor output resistance to charge a sampling capacitor typically larger than Cs′ to the full value of sensor 410's output voltage Vso′, the sampling is done in 2 stages. The two stages include a low value sampling capacitor Cs′ and a typical value hold capacitor Cₕ′. The value of capacitor Cₕ′ is chosen to be large enough such that current leakages and charge injection from measurement have minimal impact on the charge stored over the time interval which mean pressure is captured. In one embodiment, the strobe duration is 0.1 microsecond, the strobe interval is 50 milliseconds, the pressure sensor output resistance is 10 Kohm, Cs′ is 0.5 pF, and Cₕ′ is 50 pF. With these parameters the time constant is approximately 5 seconds resulting in a low pass filter 3 dB corner of approximately 0.032 Hz.

In operation, when switch S₁′ is closed, pressure sensor 410 is strobed on and obtains a pressure measurement, capacitor Cs′ is fully charged to the pressure sensor's 410 output voltage Vso′ using a much shorter strobe duration. When connected in parallel with hold capacitor Cₕ′ a low pass filter effect is created for the voltage on the sampling capacitor Cs′. When charge is transferred with small capacitor Cs′ via a non-overlapping clock that controls S₂′ and S₃′ in repeating sequence, the effect is that of a large resistor which, in combination with Cs′, has a low pass filter effect.

In one embodiment, implantable monitoring device 450 is used to monitor intracardiac blood pressure. In another embodiment, implantable monitoring device 450 is used to monitor vascular blood pressure. In one embodiment, monitoring device 450 is used to monitor blood pressure for the detection and/or treatment of hypertension, syncope, congestive heart failure and the like. In an alternate embodiment, implantable device 400 further includes an implantable therapy device 425. Implantable therapy device 425 is a pacemaker, defibrillator, drug infusion pump or the like. In one embodiment, in combination, monitoring device 450 qualifies the need for therapy by measuring a relative parameter of blood pressure. For example, in one embodiment the therapy device 425 is a defibrillator and when therapy device 425 determines that the heart needs defibrillating the monitoring device confirms the presence of an aberrant heart rhythm by measuring a relative blood pressure parameter. When both the therapy device 425 and the monitoring device 450 indicate aberrant heart rhythm therapy is administered.

In one embodiment, conversion circuitry 435 records data collected such as the value of the charge of sampling capacitor Cs′, hold capacitor Cₕ′, and the like and stores it in memory. In another embodiment, conversion circuitry 435 wirelessly transmits data collected to remote circuitry for analysis and/or recording.

In alternate embodiments the intracardiac ECG waveform, which is normally acquired by a therapy device, is used to trigger when to sample the pressure waveform such as at an anticipated pressure minimum or maximum. In an alternate embodiment, the ECG is used in combination with sampling techniques to identify beginning and end points of the cardiac cycle such that the mean pressures are calculated over individual cardiac cycles rather than as a continuous running mean over many cardiac cycles. This gives a more immediate result, which is important in certain types of cardiac therapy such as defibrillation.

In one embodiment, in order to mitigate measurement error or drift caused by leakage or charge injection is to occasionally sample the pressure sensor with standard full-length strobes to get an accurate measurement that is compared to the reduced-power methods. The difference between the two types of measurement is then used to correct the reduced-power measurements on an ongoing basis.

FIG. 5 includes a graph showing a pressure waveform 530 and an ECG waveform 520 plotted along a common time axis. In FIG. 5 the amplitude of pressure waveform 530 is measured in mn fHg. ECG waveform 520 is shown having an amplitude in the millivolt range in FIG. 5. ECG waveform 520 includes a P-wave, a QRS complex and a T-wave. In the embodiment of FIG. 5, a first delay 580 is shown extending between a first fiduciary point 584 in ECG waveform 520 and a maximum point 588 of pressure waveform 530. In the exemplary embodiment of FIG. 5, first fiduciary point 584 corresponds with the end of the QRS complex of ECG waveform 520.

Some methods in accordance with the present invention may include the steps of detecting a fiduciary point in an ECG waveform and transmitting a signal requesting a pressure measurement after a predefined delay. In some applications, the predefined delay may be determined based on a desired pressure to be measured. In some exemplary embodiments, the predefined delay approximates the point in time at which maximum blood pressure occurs and the fiduciary point comprises an endpoint of a QRS complex. In some such embodiments, the predefined delay may be between about 0.05 seconds and about 0.35 seconds. In some of these embodiments, the predefined delay may be between about 0.10 seconds and about 0.25 seconds. Also in some of these embodiments the predefined delay may be about 0.15 seconds.

In FIG. 5, a second delay 582 is shown extending between first fiduciary point 584 in ECG waveform 520 and a minimum point 590 of pressure waveform 530. Some methods in accordance with the present invention may include the steps of detecting a fiduciary point comprising the end of a QRS complex in an ECG waveform and transmitting a signal requesting a pressure measurement after a predefined delay that approximates the point in time at which minimum blood pressure occurs. In some such embodiments, the predefined delay may be between about 0.16 seconds and about 0.56 seconds. In some of these embodiments, the predefined delay may be between about 0.26 seconds and about 0.46 seconds. Also in some of these embodiments the predefined delay may be about 0.36 seconds.

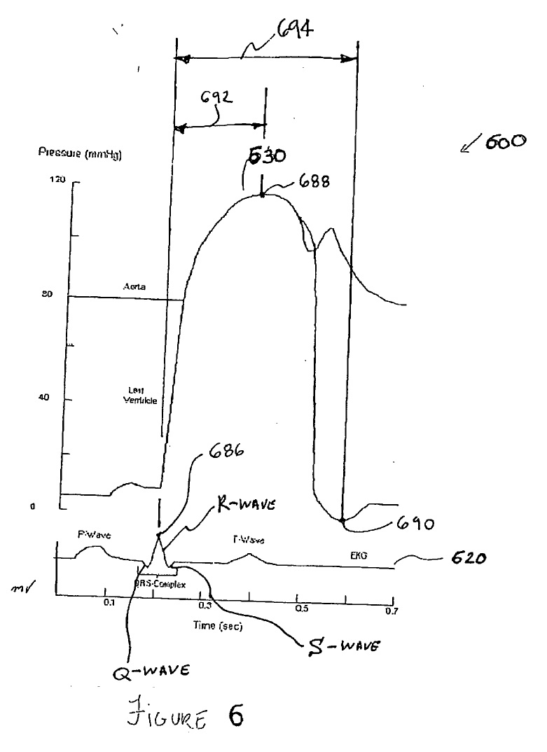

FIG. 6 includes an additional graph showing a pressure waveform 630 and an ECG waveform 620 plotted along a common time axis. ECG waveform 620 of FIG. 6 includes a P-wave, a QRS complex and a T-wave. The QRS complex comprises a Q wave, an R wave, and an S wave. In FIG. 6 a secondary fiduciary point 686 is shown overlaying the peak of the R wave in ECG waveform 620. Also in FIG. 6, a third delay 692 is shown extending between second fiduciary point 686 and a maximum point 688 of pressure waveform 630.

Some methods in accordance with the present invention may include the steps of detecting a fiduciary point in an ECG waveform and transmitting a signal requesting a pressure measurement after a predefined delay. In some applications, the predefined delay may be determined based on a desired pressure to be measured. In some exemplary embodiments, the predefined delay approximates the point in time at which maximum blood pressure occurs and the fiduciary point comprises the peak of an R wave. In some such embodiments, the predefined delay may be between about 0.07 seconds and about 0.37 seconds. In some of these embodiments, the predefined delay may be between about 0.12 seconds and about 0.27 seconds. Also in some of these embodiments the predefined delay may be about 0.17 seconds.

In FIG. 6, a fourth delay 694 is shown extending between second fiduciary point 686 in ECG waveform 620 and a minimum point 690 of pressure waveform 630. Some methods in accordance with the present invention may include the steps of detecting a fiduciary point comprising the peak of an R wave in an ECG waveform and transmitting a signal requesting a pressure measurement after a predefined delay that approximates the point in time at which minimum blood pressure occurs. In some such embodiments, the predefined delay may be between about 0.18 seconds and about 0.58 seconds. In some of these embodiments, the predefined delay may be between about 0.28 seconds and about 0.48 seconds. Also in some of these embodiments the predefined delay may be about 0.38 seconds.

Although specific embodiments have been illustrated and described herein, it will be appreciated by those of ordinary skill in the art that any arrangement, which is calculated to achieve the same purpose, may be substituted for the specific embodiments shown. This application is intended to cover any adaptations or variations of the present invention. Therefore, it is intended that this invention be limited only by the claims and the equivalents thereof.

What is claimed is:

1. A method of reducing power in a pressure monitoring device, the method comprising:
monitoring an electrocardiogram (ECG) waveform;
detecting a fiduciary point in the ECG waveform;
transmitting a signal requesting a pressure measurement, after a predefined delay, wherein the predefined delay is determined based on the desired pressure measurement and the detected fiduciary point;
applying power to a pressure sensor; and
applying an output voltage of the pressure sensor to a sampling circuit;
wherein the output voltage represents measured pressure.

2. The method of claim 1, wherein the sampling circuit comprises a sample and hold (S&H) capacitor.

3. The method of claim 2, further comprising calculating mean pressure using the measured pressure.

4. The method of claim 2, further comprising disconnecting the S&H capacitor from the sensor and removing power to the pressure sensor once voltage on the sample and hold capacitor is stable.

5. The method of claim 1, wherein detecting a fiduciary point in the ECG waveform comprises detecting a QRS complex.

6. The method of claim 5, wherein the fiduciary point comprises an end point of the QRS complex.

7. The method of claim 1, wherein the predefined delay approximates the point in time that maximum pressure occurs.

8. The method of claim 7, wherein the fiduciary point comprises an endpoint of a QRS complex and the predefined delay comprises between about 0.05 seconds and about 0.35 seconds.

9. The method of claim 8, wherein the predefined delay comprises between about 0.10 seconds and about 0.25 seconds.

10. The method of claim 9, wherein the predefined delay comprises about 0.15 seconds.

11. The method of claim 7, wherein the fiduciary point comprises a peak of an R wave and the predefined delay comprises between about 0.07 seconds and about 0.37 seconds.

12. The method of claim 11, wherein the predefined delay comprises between about 0.12 seconds and about 0.27 seconds.

13. The method of claim 12, wherein the predefined delay comprises about 0.17 seconds.

14. The method of claim 7, wherein maximum pressure corresponds to systolic pressure.

15. The method of claim 1, wherein the predefined delay approximates the point in time that minimum pressure occurs.

16. The method of claim 15, wherein minimum pressure corresponds to diastolic pressure.

17. The method of claim 15, wherein the fiduciary point comprises an endpoint of a QRS complex and the predefined delay comprises between about 0.16 seconds and about 0.56 seconds.

18. The method of claim 17, wherein the predefined delay comprises between about 0.26 seconds and about 0.46 seconds.

19. The method of claim 18, wherein the predefined delay comprises about 0.36 seconds.

20. The method of claim 15, wherein the fiduciary point comprises a peak of an R wave and the predefined delay comprises between about 0.18 seconds and about 0.58 seconds.

21. The method of claim 20, wherein the predefined delay comprises between about 0.28 seconds and about 0.48 seconds.

22. The method of claim 21, wherein the predefined delay comprises about 0.38 seconds.

23. The method of claim 1, wherein the predefined delay is based on heart rate determined from monitoring recent intervals of the ECG waveform.

24. The method of claim 1, wherein the predefined delay is adjusted based on intermittent monitoring of pressure waveforms during one or more preceding cardiac intervals.

25. The method of claim 1, wherein the fiduciary point comprises one of the P, Q, R, S, and T waveforms.

26. The method of claim 1, wherein the fiduciary point comprises an end point of one of the P, Q, R, S, and T waveforms.

27. A method of reducing power in a therapy device, the method comprising:
monitoring a physiological function;
detecting a need for an adjustment in therapy;
qualifying the need for an adjustment in therapy, including:
transmitting a signal requesting a pressure measurement based on the detected need for an adjustment in therapy;
applying power to a pressure sensor; and
measuring pressure; and
adjusting therapy when the measured pressure confirms the detected need for an adjustment in therapy.

28. The method of claim 27, wherein monitoring a physiological function comprises monitoring an intracardiac electrocardiogram waveform.

29. The method of claim 27, further comprising applying an output voltage of the pressure sensor to a sampling circuit, wherein the output voltage represents measured pressure.

30. The method of claim 27, wherein adjusting therapy comprises defibrillating a heart.

31. The method of claim 27, wherein adjusting therapy comprises resynchronizing a pacing device.

32. The method of claim 27, wherein measuring pressure comprises measuring a blood pressure waveform.

33. An implantable device, comprising:
a monitoring device configured to measure pressure for the detection of one or more heart related disorders, the device including:
a pressure sensor;
wherein the pressure sensor is strobed at a rate to create a low-pass filter effect and generate mean pressure; and
a sampling capacitor coupled to an output of the pressure sensor and adapted to charge to a voltage output that represents the mean pressure.

34. The device of claim 33, wherein the pressure sensor is a piezoresistive pressure sensor.

35. The device of claim 33, wherein the monitoring device monitors blood pressure for the treatment of one of hypertension, syncope and congestive heart failure.

36. An implantable device, comprising:
a therapy device configured to detect the need for an adjustment in therapy;
a monitoring device coupled to the therapy device, wherein the monitoring device is configured to qualify the need for the adjustment in therapy by measuring a relative parameter of blood pressure, the device including:
a pressure sensor; and
a sampling capacitor coupled to an output of the pressure sensor and adapted to charge to a voltage output of the pressure sensor;
wherein the pressure sensor is strobed at a rate to create a low-pass filter effect and generate mean pressure;
wherein the sampling capacitor is charged to the output voltage that represents the mean pressure.

37. The device of claim 36, wherein the mean pressure is the relative parameter of blood pressure used to qualify the need for therapy adjustment.

38. The device of claim 36, wherein the monitoring device monitors intracardiac blood pressure.

39. The device of claim 36, wherein the monitoring device monitors vascular blood pressure.

40. The device of claim 36, wherein the monitoring device monitors blood pressure for the treatment of one of hypertension, syncope and congestive heart failure.

41. The device of claim 36, wherein the therapy device is a pacemaker.

42. The device of claim 36, wherein the therapy device is a defibrillator.

43. The device of claim 36, wherein the therapy device is drug infusion pump.

44. The device of claim 36, wherein the pressure sensor is a piezoresistive pressure sensor.

45. A therapy device comprising:
a means for monitoring a physiological function;
a means for detecting a need for an adjustment in therapy;
a means for qualifying the need for an adjustment in therapy, including:
a means for transmitting a signal requesting a pressure measurement based on the detected need for an adjustment in therapy;
a means for applying power to a pressure sensor; and
a means for measuring pressure; and
a means for adjusting therapy when the measured pressure confirms the detected need for an adjustment in therapy.

46. The device of claim 45, wherein the means for monitoring a physiological function comprises a means for monitoring an intracardiac electrocardiogram waveform.

47. The device of claim 45, further comprising a means for applying an output voltage of the pressure sensor to a sampling circuit, wherein the output voltage represents measured pressure.

48. The device of claim 45, wherein the means for adjusting therapy comprises a means for defibrillating a heart.

49. The device of claim 45, wherein the means for adjusting therapy comprises a means for resynchronizing a pacing device.

50. The device of claim 45, wherein the means for measuring pressure comprises a means for measuring a blood pressure waveform.

51. A pressure monitoring device, comprising:
a means for monitoring an electrocardiogram (ECG) waveform;
a means for detecting a fiduciary point in the ECG waveform;
a means for transmitting a signal requesting a pressure measurement, after a predefined delay, wherein the predefined delay is determined based on the desired pressure measurement and the detected fiduciary point;
a means for applying power to a pressure sensor; and
a means for applying an output voltage of the pressure sensor to a sampling circuit;
wherein the output voltage represents measured pressure.

52. The device of claim 51, wherein the predefined delay approximates the point in time that minimum pressure occurs.

53. The device of claim 51, wherein the predefined delay is based on heart rate determined from monitoring recent intervals of the ECG waveform.

54. The device of claim 51, further comprising a means for calculating mean pressure using the measured pressure.

55. The device of claim 51, further comprising a means for disconnecting the sampling circuit from the sensor and removing power to the pressure sensor once voltage on the sampling circuit is stable.

56. The device of claim 51, wherein the means for detecting a fiduciary point in the ECG waveform comprises a means for detecting a QRS complex.

57. The device of claim 51, wherein the predefined delay approximates the point in time that maximum pressure occurs.

58. The device of claim 51, wherein the predefined delay is adjusted based on intermittent monitoring of pressure waveforms during one or more preceding cardiac intervals.

59. The device of claim 51, wherein the fiduciary point is one of the P, Q, R, S, and T waveforms.

60. An implantable device, comprising:
a monitoring device configured to measure pressure for the detection of one or more heart related disorders, the device including:
a pressure sensor;
a means for strobing the pressure sensor at a rate to create a low-pass filter effect; and
a sampling capacitor coupled to an output of the pressure sensor and adapted to charge to a voltage output that represents the mean pressure.

61. The device of claim 60, wherein the pressure sensor is a piezoresistive pressure sensor.

62. The device of claim 60, wherein the monitoring device monitors blood pressure for the treatment of one of hypertension, syncope and congestive heart failure.
